(19) United States
(12) Patent Application Publication
(10) Pub. No.: US 2022/0304747 A1
(43) Pub. Date: Sep. 29, 2022

(54) HIGH-FREQUENCY TREATMENT TOOL, MEDICAL SYSTEM, AND METHOD FOR REMOVING ATTACHED MATTER ON HIGH-FREQUENCY TREATMENT TOOL

(71) Applicant: OLYMPUS CORPORATION, Tokyo (JP)

(72) Inventors: Yasuo MIYANO, Tokyo (JP); Yoshinori HIGUCHI, Tokyo (JP); Kenji MURAKAMI, Tokyo (JP); Shohei KAMADA, Tokyo (JP); Yoshitaka KAMIYA, Tokyo (JP); Masayoshi SAITO, Tokyo (JP)

(73) Assignee: OLYMPUS CORPORATION, Tokyo (JP)

(21) Appl. No.: 17/839,702

(22) Filed: Jun. 14, 2022

Related U.S. Application Data
(63) Continuation of application No. PCT/JP2019/049125, filed on Dec. 16, 2019.

(51) Int. Cl. A61B 18/14

(52) U.S. Cl. CPC A61B 18/1492; A61B 2018/00601

(57) ABSTRACT

Provided is a high-frequency treatment tool including: a sheath having an inner hole that passes therethrough in a longitudinal direction; a first electrode portion that is formed in a rod shape, that passes through the inner hole of the sheath to protrude from a distal end of the sheath, and that is configured to apply a high-frequency current; a second electrode portion that is disposed at a position at which the second electrode portion is electrically connected with the first electrode portion; and a power source that uses the first electrode portion as a negative electrode, that uses the second electrode portion as a positive electrode, and that supply a current between the first electrode portion and the second electrode portion so that a state in which attached matter attached to the first electrode portion is lifted from the first electrode portion due to osmosis is created.

### CROSS-REFERENCE TO RELATED APPLICATIONS

This is a continuation of International Application PCT/JP2019/049125 which is hereby incorporated by reference herein in its entirety.

### TECHNICAL FIELD

The present invention relates to a high-frequency treatment tool, a medical system, and a method for removing attached matter on a high-frequency treatment tool.

### BACKGROUND ART

In the related art, there is a known high-frequency treatment tool that transendoscopically makes an incision in biological tissue such as a mucous membrane (for example, see Patent Literature 1). The high-frequency treatment tool described in Patent Literature 1 includes a rod-like electrode that protrudes from a distal end of a sheath in the longitudinal direction. The high-frequency treatment tool described in Patent Literature 1 makes a cautery incision in biological tissue by bringing the electrode into contact with the biological tissue in a state in which the electrode is energized with a high-frequency current.

With the high-frequency treatment tool described in Patent Literature 1, when a cautery incision is made in biological tissue, the incising performance thereof deteriorates as a result of a burnt deposit of the incised biological tissue becoming attached to the electrode. Accordingly, in the case in which a burnt deposit of biological tissue becomes attached to the electrode, treatment is performed by temporarily removing the high-frequency treatment tool from an endoscope channel and by inserting the high-frequency treatment tool into the endoscope channel again after removing the burnt deposit of the biological tissue from the electrode.

### CITATION LIST

### Patent Literature

{PTL 1} PCT International Publication No. WO 2014/042039

### SUMMARY OF INVENTION

One aspect of the present invention is a high-frequency treatment tool including: a sheath having an inner hole that passes therethrough in a longitudinal direction; a first electrode portion that is formed in a rod shape, that passes through the inner hole of the sheath to protrude from a distal end of the sheath, and that is configured to apply a high-frequency current; a second electrode portion that is disposed at a position at which the second electrode portion is electrically connected with the first electrode portion; and a power source that uses the first electrode portion as a negative electrode, that uses the second electrode portion as a positive electrode, and that supply a current between the first electrode portion and the second electrode portion so that a state in which attached matter attached to the first electrode portion is lifted from the first electrode portion due to osmosis is created.

Another aspect of the present invention is a method for removing attached matter on a high-frequency treatment tool, the method including: making a first electrode portion disposed in a sheath protrude from a distal end of the sheath toward a distal end, the first electrode portion being formed in a rod shape; releasing an electrolyte liquid from the distal end of the sheath toward the first electrode portion; supplying a current between the first electrode portion and a second electrode portion so that a state in which attached matter attached to the first electrode portion is lifted from the first electrode portion due to osmosis is created, the second electrode portion being disposed at a position at which the second electrode portion is electrically connected with the first electrode portion; and pulling the first electrode portion into the sheath.

### BRIEF DESCRIPTION OF DRAWINGS

FIG. 1 is an overall configuration diagram of a medical system according to a first embodiment of the present invention.

FIG. 2 is an overall configuration diagram of a high-frequency treatment tool in FIG. 1 when excising tissue.

FIG. 3 is an overall configuration diagram of the high-frequency treatment tool in FIG. 2 when removing a burnt deposit of the biological tissue.

FIG. 4 is a flowchart for explaining a high-frequency treatment tool operating method employing the high-frequency treatment tool in FIG. 2.

FIG. 5 is an overall configuration diagram of a medical system according to a modification of the first embodiment of the present invention.

FIG. 6 is a diagram for explaining a manner in which a high-frequency current is biased toward a negative side.

FIG. 7 is an overall configuration diagram of a high-frequency treatment tool according to a second embodiment of the present invention when excising tissue.

FIG. 8 is an overall configuration diagram of the high-frequency treatment tool in FIG. 7 when removing a burnt deposit of the biological tissue.

FIG. 9 is a longitudinal cross-sectional view showing the vicinity of a sheath distal-end portion of a high-frequency treatment tool according to a first modification of the second embodiment of the present invention.

FIG. 10 is a side view showing the vicinity of a sheath distal-end portion, which is a further modification of the high-frequency treatment tool according to the first modification of the second embodiment of the present invention.

FIG. 11 is a longitudinal cross-sectional view of the vicinity of the sheath distal-end portion in FIG. 10.

FIG. 12 is a cross-sectional view taken across A-A in FIG. 10.

FIG. 13 is a cross-sectional view taken across B-B in FIG. 10.

FIG. 14 is a perspective view showing a cutter in FIG. 13.

FIG. 15 is a longitudinal cross-sectional view showing a state in which an electrode portion of the high-frequency treatment tool in FIG. 10 is pulled into a sheath.

FIG. 16 is a side view showing the vicinity of the sheath distal-end portion of the high-frequency treatment tool according to the second modification of the second embodiment of the present invention.

FIG. 17 is a longitudinal cross-sectional view of the vicinity of the sheath distal-end portion in FIG. 16.

FIG. 18 is a cross-sectional view taken across D-D in FIG. 16.

FIG. 19 is an enlarged view of the sheath distal-end portion in FIG. 17.

FIG. 20 is a cross-sectional view taken across C-C in FIG. 16.

FIG. 21 is a longitudinal cross-sectional view showing a state in which an electrode portion of the high-frequency treatment tool in FIG. 16 is pulled into a sheath.

### DESCRIPTION OF EMBODIMENTS

### First Embodiment

A high-frequency treatment tool, a medical system, and a high-frequency treatment tool operating method according to a first embodiment of the present invention will be described below with reference to the drawings.

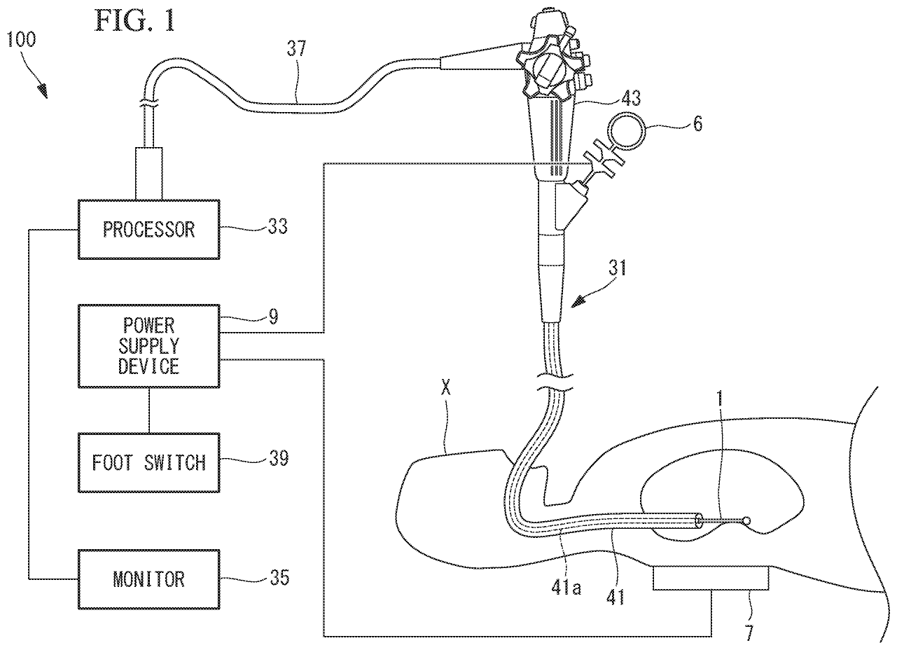

As shown in FIG. 1, a medical system 100 according to this embodiment includes: a flexible endoscope 31; a high-frequency treatment tool 1 that makes an incision in biological tissue of a patient (subject) X; a processor 33 that performs tasks such as overall control of the medical system 100 and endoscope image generation; and so forth. In FIG. 1, reference sign 35 indicates a monitor that displays an endoscope image or the like generated by the processor 33. In addition, reference sign 37 indicates a universal cable that connects the endoscope 31 and the high-frequency treatment tool 1 to the processor 33.

The endoscope 31 includes a long, thin insertion portion 41 that can be inserted into a body of a patient X (into a living body and an endoscope operating portion 43 for operating the insertion portion 41, feeding of air and liquids, endoscope image acquisition, and so forth.

The insertion portion 41 is provided with a channel 41a into which the high-frequency treatment tool 1 can be inserted.

The high-frequency treatment tool 1 passes through the channel 41a of the endoscope 31, and a distal end thereof is introduced into the body of the patient X. As shown in FIGS. 1-3, the high-frequency treatment tool 1 includes: a long, thin cylindrical sheath 3 possessing flexibility; a knife portion 5 that is moved forward and rearward at a distal end of the sheath 3; a knife operating portion 6 for performing operations such as changing the protrusion amount of the knife portion 5; an opposing electrode (second electrode portion) 7 that is disposed outside the body of the patient X; a power supply device 9 that supplies currents to the knife portion 5 and the opposing electrode 7; and a liquid feeding means 11 that supplies a physiological saline solution (liquid) W between the knife portion 5 and the opposing electrode 7. In the following, a distal-end side of the sheath 3 is assumed to be forward, and a basal-end side of the sheath 3 is assumed to be rearward.

The sheath 3 is formed so as to allow the insertion thereof into the channel 41a of the endoscope 31. The sheath 3 includes, for example, a cylindrical coil (not shown) that has an inner hole 3a that passes therethrough in a longitudinal direction and a cylindrical insulation tube (not shown) that covers an outer circumference of the coil. The inner hole 3a also serves as a flow channel of the liquid. The liquid feeding means 11 is a syringe, a pump, or the like that is connected to the inner hole 3a, and the physiological saline solution W is released from the distal end of the sheath 3 via the inner hole 3a.

The knife portion 5 includes: an electrode portion (first electrode portion) 13 that can be made to protrude from the distal end of the sheath 3 by passing through the inner hole 3a of the sheath 3; and a substantially hemispherical distal-end tip 15 that is secured to a distal end of the electrode portion 13.

The electrode portion 13 includes: a needle 13a which is a rod-like electrode having a constant diameter over the entire length thereof; and an electrode 13b provided at a distal end of the needle 13a.

The needle 13a is provided so as to be relatively movable in the inner hole 3a of the sheath 3 in the longitudinal direction of the sheath 3. The movement of the needle 13a is controlled by the knife operating portion 6. The needle 13a is formed of, for example, a conductive material such as SUS (stainless steel).

The electrode 13b is formed of, for example, a conductive material such as SUS, as with the needle 13a, and is integrally formed at the distal end of the needle 13a. The electrode 13b extends, for example, from the distal end of the needle 13a in a radiating manner in a direction orthogonal to the longitudinal-axis direction of the needle 13a.

The distal-end tip 15 is formed of, for example, a heat-resistant electrical insulator such as a ceramic. The distal-end tip 15 is disposed, for example, with a spherical surface portion 15a thereof facing away from the sheath 3 and a flat surface portion 15b facing toward the sheath 3. The electrode 13b is secured to the flat surface portion 15b, and the electrode 13b extends in a radiating manner along the flat surface portion 15b.

The knife operating portion 6 is disposed on the basal-end side of the sheath 3. The knife operating portion 6 includes, for example, an operating portion body that has a longitudinal axis, an operating slider that is provided in the operating portion body so as to be movable in the longitudinal-axis direction of the operating portion body, and an operating wire that connects the operating slider and the knife portion 5 (all of which are not shown).

The operating wire is disposed inside the inner hole 3a of the sheath 3, a distal end thereof is connected to the basal-end portion of the needle 13a and a basal end thereof is connected to the operating slider. When the operating slider is moved in the longitudinal-axis direction of the operating portion body, a pressing force and a pulling force are transmitted to the needle 13a as a result of the operating wire being pushed and pulled in the longitudinal direction of the sheath 3. Accordingly, the needle 13a is moved with respect to the sheath 3 in the longitudinal direction of the sheath 3. In other words, the knife portion 5 is moved forward and rearward with respect to the sheath 3 in association with the forward and rearward motions of the operating wire.

The opposing electrode 7 is formed of a conductive material such as SUS, as with the needle 13a and the electrode 13b. The opposing electrode 7 is attached to, for example, the back of the patient X. Note that the material of the needle 13a, the electrode 13b, and the opposing electrode 7 is not limited to SUS, and all of these components may be made of any conductive material.

The power supply device 9 includes: a high-frequency power source 17 that supplies high-frequency currents between the electrode portion 13 and the opposing electrode 7; a constant-current DC power source 19 that supplies direct currents between the electrode portion 13 and the opposing electrode 7; and a switching mechanism 21 that switches between the high-frequency current supply between the electrode portion 13 and the opposing electrode 7 and the direct current supply therebetween. A foot switch 39 for an operator to control the high-frequency power source 17, the constant-current DC power source 19, and the switching mechanism 21 is connected to the power supply device 9 (see FIG. 1).

The switching mechanism 21 includes: a first switch 21a that connects the needle 13a to one of a knife-side terminal 17a of the high-frequency power source 17 and a negative electrode terminal (−) 19b of the constant-current DC power source 19 in a switchable manner; and a second switch 21b that connects the opposing electrode 7 to one of an opposing-electrode-side terminal 17b of the high-frequency power source 17 and a positive electrode terminal (+) 19a of the constant-current DC power source 19 in a switchable manner.

Next, the operation of the high-frequency treatment tool 1 and the medical system 100 according to this embodiment will be described below.

In order to transendoscopically excise a mucous membrane in a body by using the medical system 100 according to this embodiment, first, an injection needle (not shown) is introduced into the body of a patient X via the channel 41a of the endoscope 31. Then, a lesion site is lifted up by injecting a physiological saline solution into a submucosa of a site that is assumed to be a lesion to be excised, while viewing an endoscope image displayed on the monitor 35.

Next, a high-frequency treatment tool (not shown) having a conventional needle-like electrode is introduced into the body via the channel 41a of the endoscope 31, and an initial incision is made, the initial incision making a hole in a portion of the mucous membrane in the periphery of the lesion site. After making the initial incision, the high-frequency treatment tool is removed from the channel 41a.

Subsequently, an operator switches the tool in hand with the high-frequency treatment tool 1 and introduces the sheath 3 into the body from the distal-end side thereof via the channel 41a of the endoscope 31, as shown in FIG. 1, in a state in which the knife portion 5 is maximally moved rearward. Because the distal-end tip 15, which is disposed at the distal end of the sheath 3, comes into the viewing field of the endoscope 31 when the distal end of the sheath 3 is made to protrude from the distal end of the channel 41a of the endoscope 31, the operator performs treatment while checking an endoscope image acquired by means of the endoscope 31 on the monitor 35.

In the state in which the knife portion 5 is maximally moved rearward, only the distal-end tip 15 is exposed from the distal end of the sheath 3; therefore, the knife portion 5 is not deeply inserted into biological tissue S. In addition, because the spherical surface portion 15a of the substantially hemispherical distal-end tip 15 is disposed facing forward, the biological tissue S that comes into contact with the distal-end tip 15 is not damaged.

Next, the knife portion 5 is maximally moved forward by means of the knife operating portion 6. Doing so puts the needle 13a and the electrode 13b in a state of being exposed forward with respect to the sheath 3. In this state, the knife portion 5 is inserted, from the distal-end tip 15, into the hole formed in advance by the initial incision.

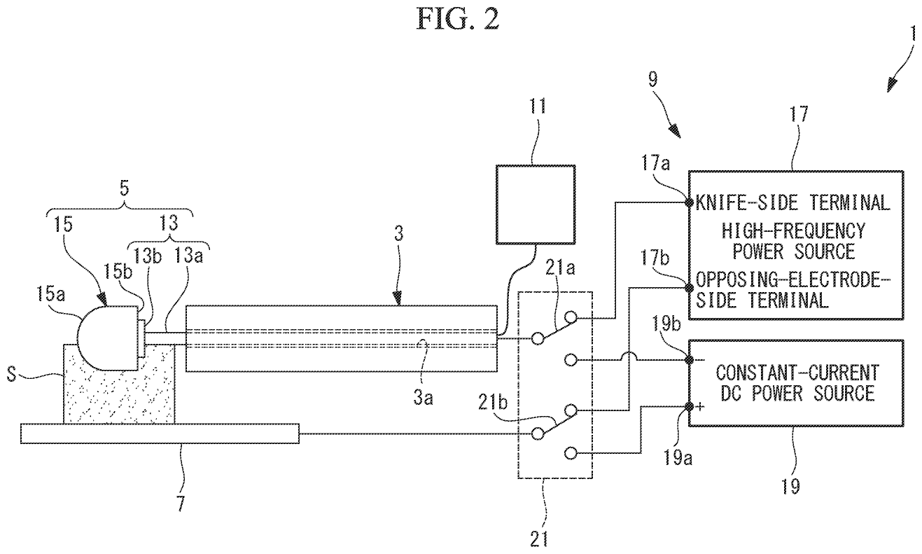

Next, as shown in FIG. 2, the needle 13a and the knife-side terminal 17a of the high-frequency power source 17 are connected by means of the first switch 21a, and the opposing electrode 7 and the opposing-electrode-side terminal 17b of the high-frequency power source 17 are connected by means of the second switch 21b.

In this state, the knife portion 5 is moved in a direction in which an incision is made, intersecting the longitudinal axis, while supplying the high-frequency currents between the needle 13a and the opposing electrode 7 as well as between the electrode 13b and the opposing electrode 7 from the high-frequency power source 17. For example, by hooking a section from the distal-end portion of the needle 13a to the electrode 13b on the mucous membrane in the periphery of the lesion site, it is possible to efficiently make a cautery incision in the periphery of the lesion site.

Because the distal-end tip 15 provided at the distal end of the knife portion 5 is formed of a material having an insulating property, an incision is not made in the biological tissue S that is in contact with the distal-end tip 15, even if the high-frequency currents are supplied to the needle 13a and the electrode 13b. Therefore, it is possible to prevent the problem of the distal-end tip 15 making an incision in submucosal tissue.

In this case, while the cautery incision is being made in the biological tissue S, burnt deposits (attached matter) of the incised biological tissue S become attached to the needle 13a and the electrode 13b. When the burnt deposits of the biological tissue S become attached to the needle 13a and the electrode 13b, the incising performance of the electrode portion 13 deteriorates; therefore, it is necessary to remove the burnt deposits of the biological tissue S from the needle 13a and the electrode 13b.

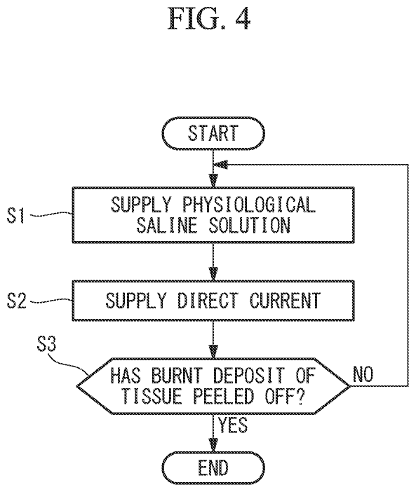

A method for operating the high-frequency treatment tool 1 for removing the burnt deposits of the biological tissue S attached to the needle 13a and the electrode 13b will be described below with reference to the flowchart in FIG. 4.

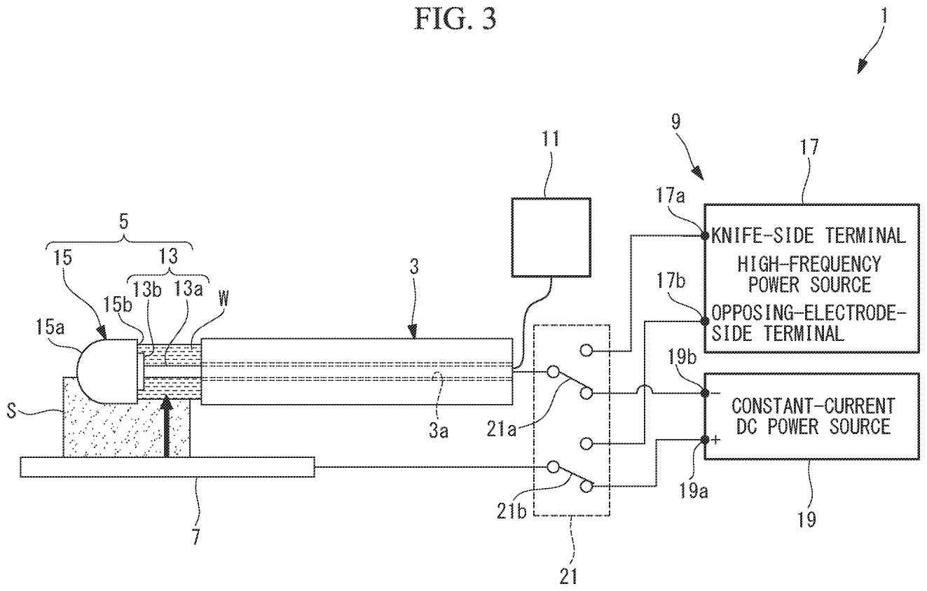

In the case in which burnt deposits of the biological tissue S become attached to the needle 13a and the electrode 13b, first, the liquid feeding means 11 is activated in the state in which the distal end of the sheath 3 remains inserted inside the body via the channel 41a of the endoscope 31. Consequently, the physiological saline solution W is released to the periphery of the electrode portion 13 from the distal end of the sheath 3, as shown in FIG. 3 (step S1). Accordingly, the needle 13a and the biological tissue S as well as the electrode 13b and the biological tissue S are electrically connected as a result of the physiological saline solution W being interposed therebetween.

Next, as shown in FIG. 3, the needle 13a and the negative electrode terminal 19b of the constant-current DC power source 19 are connected by means of the first switch 21a, and the opposing electrode 7 and the positive electrode terminal 19a of the constant-current DC power source 19 are connected by means of the second switch 21b. In this state, the direct currents are supplied between the needle 13a and the opposing electrode 7 as well as between the electrode 13b and the opposing electrode 7 from the constant-current DC power source 19 (step S2).

Consequently, the physiological saline solution W moves to the periphery of the electrode portion 13 due to osmosis. Specifically, the physiological saline solution W permeates the burnt deposits of the biological tissue S attached to the needle 13a and the electrode 13b and collects in the periphery of the needle 13a and the electrode 13b. Accordingly, a state in which the burnt deposits of the biological tissue S attached to the needle 13a and the electrode 13b are lifted from the needle 13a and the electrode 13b is created, and thus, it becomes easier for the burnt deposits of the biological tissue S to peel off from the needle 13a and the electrode 13b.

In the case in which the burnt deposits of the biological tissue S are removed from the needle 13a and the electrode 13b (“YES” in step S3), the removal processing of the burnt deposits of the biological tissue S is ended, and the treatment is restarted.

On the other hand, in the case in which the burnt deposits of the biological tissue S are not removed from the electrode portion 13 (“NO” in step S3), steps S1 and S2 are repeated until the burnt deposits of the biological tissue S are removed from the electrode portion 13.

As has been described above, with the high-frequency treatment tool 1 and the method for operating the high-frequency treatment tool 1 according to this embodiment, in the case in which the burnt deposits of the biological tissue S become attached to the needle 13a and the electrode 13b, it is possible to remove the burnt deposits of the biological tissue S from the needle 13a and the electrode 13b in a state in which the sheath 3 remains inserted in the channel 41a of the endoscope 31 simply by supplying the direct currents between the needle 13a and the opposing electrode 7 as well as between the electrode 13b. and the opposing electrode 7.

Therefore, even if burnt deposits of the biological tissue S become attached to the needle 13a and the electrode 13b, it is possible to enhance the work efficiency by reducing the time and effort required to remove the high-frequency treatment tool 1 from the channel 41a of the endoscope 31. In addition, it is possible to share the opposing electrode 7 between when making an incision in the biological tissue S and when removing the burnt deposits of the biological tissue S attached to the electrode portion 13, and thus, it is possible to reduce the number of components.

In this embodiment, the high-frequency currents and the direct currents are switched; however, alternatively, for example, the high-frequency currents and the direct currents may be applied to the electrode portion 13 in an overlapping manner. In the case in which the high-frequency currents and the direct currents are overlapped, the two types of currents may be constantly overlapped or may be overlapped after applying the high-frequency currents.

Regarding the direct currents, it suffices, so long as the capacitance thereof is high enough, to apply the negative bias required to cause the burnt deposits of the biological tissue S attached to the electrode portion 13 to peel off therefrom.

It is possible to modify this embodiment as in the following configuration.

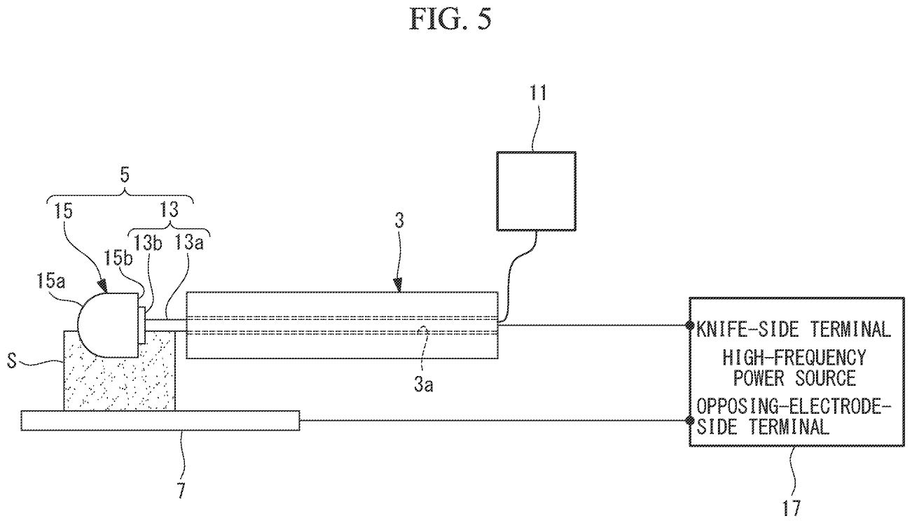

A high-frequency treatment tool 1 according to the modification of this embodiment consists of, for example, the sheath 3, the knife portion 5, the opposing electrode 7, the liquid feeding means 11, and the high-frequency power source 17, as shown in FIG. 5. The sheath 3, the knife portion 5, the opposing electrode 7, and the liquid feeding means 11 are configured in the same manner as in the first embodiment. The positive side of the high-frequency power source 17 is directly connected to the needle 13a without passing through the switching mechanism 21, and the negative side thereof is directly connected to the opposing electrode 7 without passing through the switching mechanism 21.

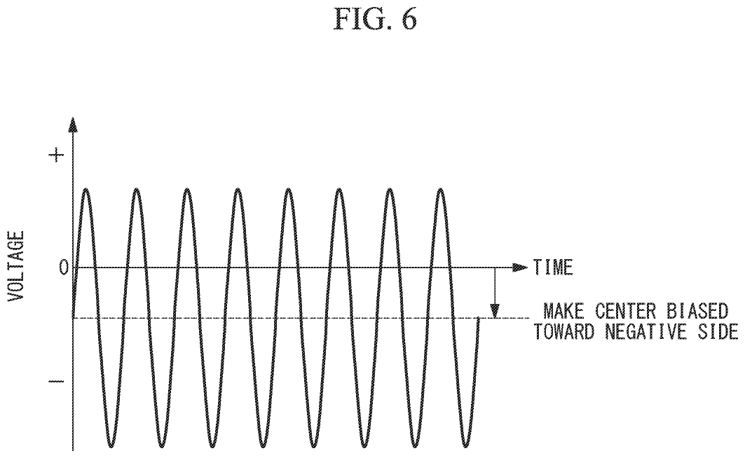

As shown in FIG. 6, the direct currents are made to overlap with the high-frequency currents. For example, the high-frequency currents are biased toward the negative side. Accordingly, because the time during which a negative volage is applied to the needle 13a increases, the needle 13a effectively behaves in the same manner as when being negatively charged. Therefore, an equivalent effect as when the direct currents are applied is achieved.

With this modification, because the equivalent effect as when the direct currents are applied is achieved by means of the configuration of the high-frequency treatment tool itself, an additional constituent component is not required, and thus, it is possible to reduce costs.

### Second Embodiment

Next, a high-frequency treatment tool, a medical system, and a high-frequency treatment tool operating method according to a second embodiment of the present invention will be described.

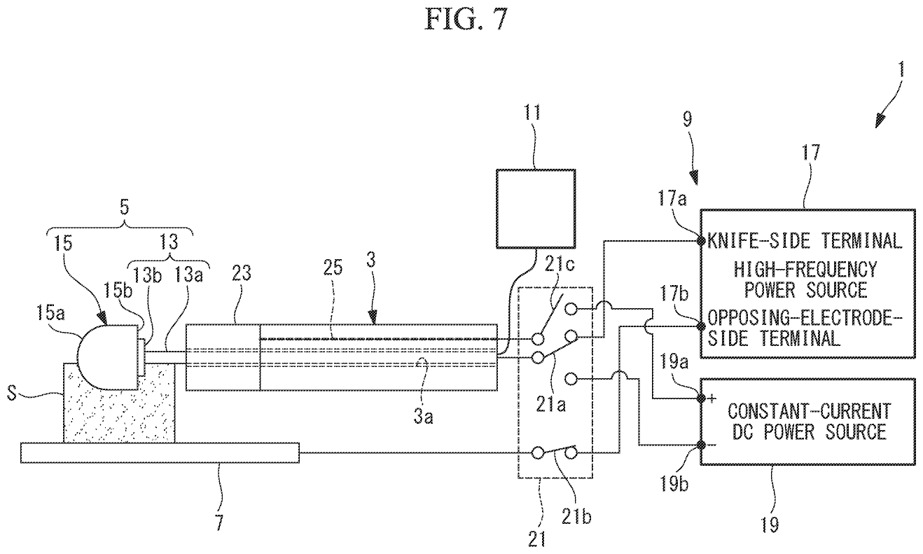

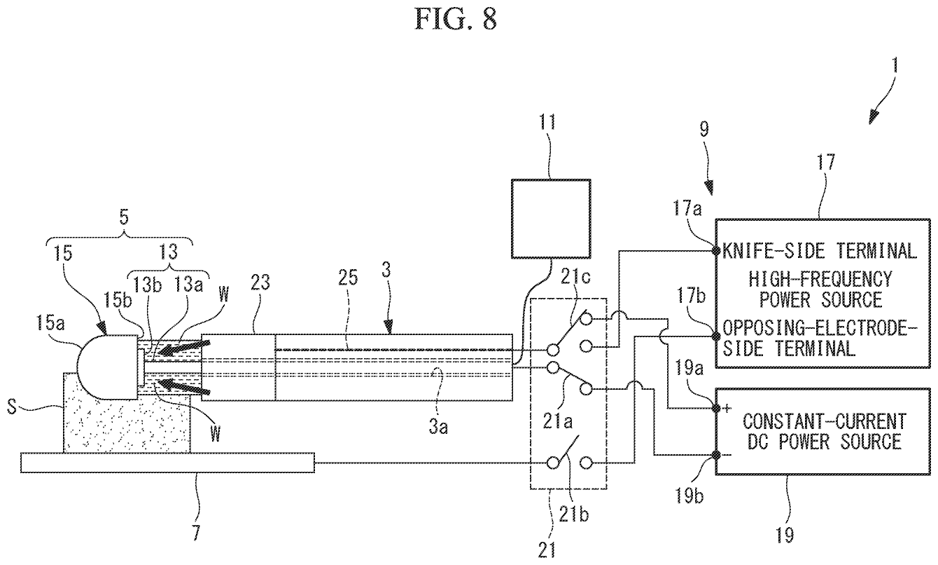

A high-frequency treatment tool 1 according to this embodiment includes, for example, as shown in FIGS. 7 and 8, a DC electrode (second electrode portion) 23 as a separate component from the opposing electrode 7, and differs from the first embodiment in that the DC electrode 23 is disposed in the distal-end portion of the sheath 3.

In the following, the portions having the same configurations as the high-frequency treatment tool 1 according to the first embodiment will be given the same reference signs, and the descriptions thereof will be omitted. The other configurations of the medical system 100 are the same as those in the first embodiment.

The DC electrode 23 is disposed at a position where the DC electrode 23 covers the outer circumference of the sheath 3 in the distal-end portion of the sheath 3. Wiring 25 for supplying power to the DC electrode 23 is disposed inside the sheath 3. The DC electrode 23 and the wiring 25 are electrically connected with each other. The DC electrode 23 is formed of, for example, a conductive material such as SUS.

In this embodiment, the switching mechanism 21 is provided with a third switch 21c that switches between connection and disconnection between the wiring 25 of the DC electrode 23 and the positive electrode terminal 19a of the constant-current DC power source 19. The second switch 21b switches between connection and disconnection between the opposing electrode 7 and the opposing-electrode-side terminal 17b of the high-frequency power source 17.

Next, the operation of the high-frequency treatment tool 1 according to this embodiment will be described below.

In the case in which a mucous membrane in a body is transendoscopically excised by using the high-frequency treatment tool 1 according to this embodiment, as shown in FIG. 7, the needle 13a and the knife-side terminal 17a of the high-frequency power source 17 are connected by means of the first switch 21a, and the opposing electrode 7 and the opposing-electrode-side terminal 17b of the high-frequency power source 17 are connected by means of the second switch 21b. On the other hand, the wiring 25 of the DC electrode 23 and the positive electrode terminal 19a of the constant-current DC power source 19 are put into a disconnected state by means of the third switch 21c, thereby putting the DC electrode 23 into an electrically floating state.

In this state, as a result of moving the knife portion 5 in the incising direction, intersecting the longitudinal axis, while supplying high-frequency currents between the needle 13a and the opposing electrode 7 as well as between the electrode 13b and the opposing electrode 7 from the high-frequency power source 17, a cautery incision is made in the periphery of a lesion site.

Next, in the case in which burnt deposits of the biological tissue S become attached to the needle 13a and the electrode 13b, the physiological saline solution W is released to the periphery of the electrode portion 13 from the distal end of the sheath 3 by means of the liquid feeding means 11, as shown in FIG. 8, in the state in which the distal end of the sheath 3 remains inserted inside the body via the channel 41a of the endoscope 31. Accordingly, the needle 13a and the DC electrode 23 as well as the electrode 13b and the DC electrode 23 are electrically connected as a result of the physiological saline solution W being interposed therebetween.

Next, the needle 13a and the negative electrode terminal 19b of the constant-current DC power source 19 are connected by means of the first switch 21a, and the wiring 25 of the DC electrode 23 and the positive electrode terminal 19a of the constant-current DC power source 19 are connected by means of the third switch 21c. On the other hand, the opposing electrode 7 and the opposing-electrode-side terminal 17b of the high-frequency power source 17 are put into a disconnected state by means of the second switch 21b, thereby putting the opposing electrode 7 into an electrically floating state.

In this state, direct currents are supplied between the needle 13a and the DC electrode 23 as well as between the electrode 13b and the DC electrode 23 from the constant-current DC power source 19. Consequently, the physiological saline solution W in the periphery of the electrode portion 13 permeates burnt deposits of the biological tissue S attached to the needle 13a and the electrode 13b and collects in the periphery of the needle 13a and the electrode 13b due to osmosis. Accordingly, it becomes easier for the burnt deposits of the biological tissue S to peel off from the electrode portion 13.

In the case in which burnt deposits of the biological tissue S are removed in this embodiment, as a result of applying the direct currents to the DC electrode 23 disposed in the distal-end portion of the sheath 3, instead of the opposing electrode 7, the direct currents are concentrated in the periphery of the electrode portion 13; therefore, it is possible to reduce the amount of current flowing inside the body.

It is possible to modify this embodiment as in the following configurations.

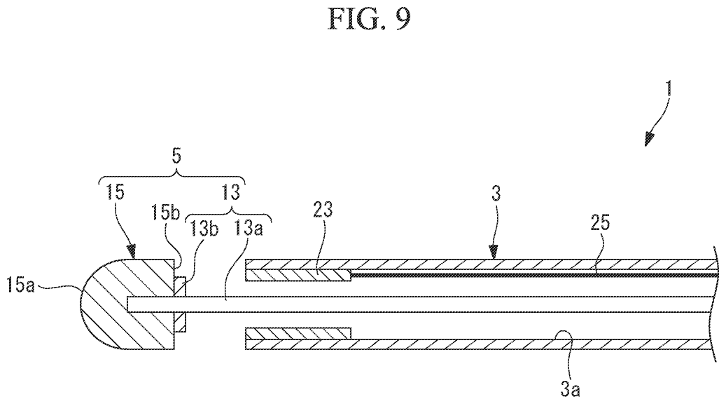

In this embodiment, the DC electrode 23 is disposed at the position where the DC electrode 23 covers the distal-end portion of the sheath 3. As a first modification, for example, the DC electrode 23 may be accommodated in the distal-end portion of the sheath 3, as shown in FIG. 9. The DC electrode 23 is formed in a tubular shape and is secured to an inner surface of the inner hole 3a of the sheath 3.

With this modification, as a result of the DC electrode 23 being accommodated in the distal-end portion of the sheath 3, when excising a mucous membrane in a body, in other words, when applying a high-frequency current to the knife portion 5, it is unlikely that the DC electrode 23 comes into contact with the biological tissue S. Therefore, an unnecessary discharge resulting from the DC electrode 23 coming into contact with the biological tissue S is prevented, and thus, it is possible to prevent a deterioration in the incising performance.

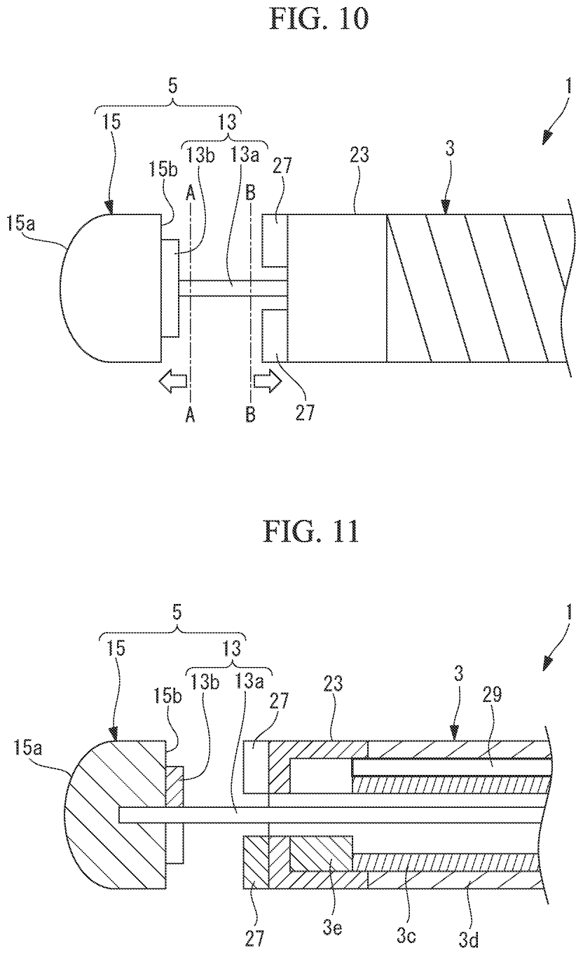

As a second modification, for example, the high-frequency treatment tool 1 may include cutters 27 disposed at the distal-end portion of the DC electrode 23, as shown in FIGS. 10 and 11. Each of the cutters 27 is disposed so that a cutting edge 27a thereof points toward the electrode portion 13.

In the example shown in FIG. 11, the sheath 3 consists of a cylindrical coil 3c having the inner hole 3a, a cylindrical tube 3d that covers an outer circumference of the coil 3c, and a cylindrical sheath distal-end member 3e that is disposed forward with respect to the coil 3c and the tube 3d.

The coil 3c is formed of, for example, a conductive material such as SUS. The tube 3d is formed of, for example, an insulator such as PTFE (polytetrafluoroethylene). The sheath distal-end member 3e is formed of, for example, an insulator such as a ceramic.

A DC power supply cable 29 that is electrically connected to the DC electrode 23 is disposed between the tube 3d and the coil 3c. The DC power supply cable 29 is covered with an insulation coating.

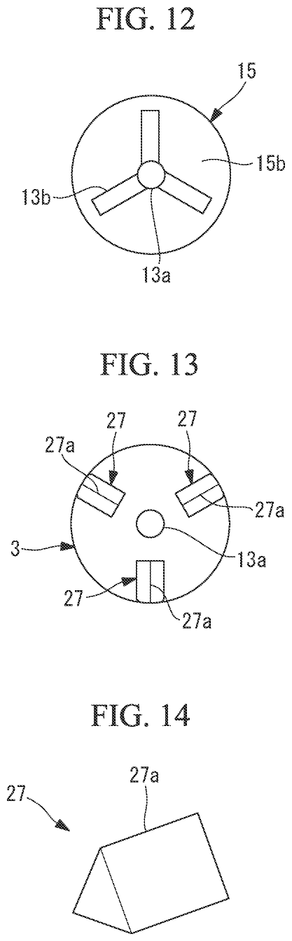

In this modification, the needle 13a is provided so as to be relatively movable in the longitudinal direction of the sheath 3. The electrode 13b extends, for example, in a Y-shape along the flat surface portion 15b of the distal-end tip 15 with equal spacings in the circumferential direction about the longitudinal axis of the needle 13a, as shown in FIG. 12, and is secured to the flat surface portion 15b.

Each of the cutters 27 is, for example, a triangular prism-shaped member and an angular portion thereof formed by two adjacent side surfaces forms the cutting edge 27a, as shown in FIGS. 13 and 14. The cutter 27 has the cutting edge 27a extending in a radial direction of the sheath 3 and is secured to a distal-end surface of the sheath 3 in an orientation in which the cutting edge 27a faces forward with respect to the sheath 3. In the example shown in FIG. 13, three cutters 27 are disposed at positions shifted in the circumferential direction about the longitudinal axis of the needle 13a with respect to the electrode 13b extending in the Y-shape.

The operation of the high-frequency treatment tool 1 according to this modification will be described below.

In the case in which burnt deposits of the biological tissue S become attached to the electrode portion 13, the physiological saline solution W is released to the periphery of the electrode portion 13 from the distal end of the sheath 3 in the state in which the distal end of the sheath 3 remains inserted inside the body via the channel 41a of the endoscope 31, and the needle 13a and the DC electrode 23 as well as the electrode 13b and the DC electrode 23 are electrically connected.

Next, the needle 13a is used as a negative electrode, the DC electrode 23 is used as a positive electrode, and direct currents are supplied between the needle 13a and the DC electrode 23 as well as between the electrode 13b and the DC electrode 23 from the constant-current DC power source 19. Consequently, the physiological saline solution W in the periphery of the electrode portion 13 permeates burnt deposits of the biological tissue S attached to the needle 13a and the electrode 13b and collects in the periphery of the needle 13a and the electrode 13b due to osmosis, as a result of which it becomes easier for the burnt deposits of the biological tissue S to peel off from the electrode portion 13.

Here, although the direct current application creates a state in which the burnt deposits of the biological tissue S are lifted from the needle 13a and the electrode 13b, there are cases in which the burnt deposits of the biological tissue S do not peel off and remain in a tubular shape around the needle 13a and the electrode 13b.

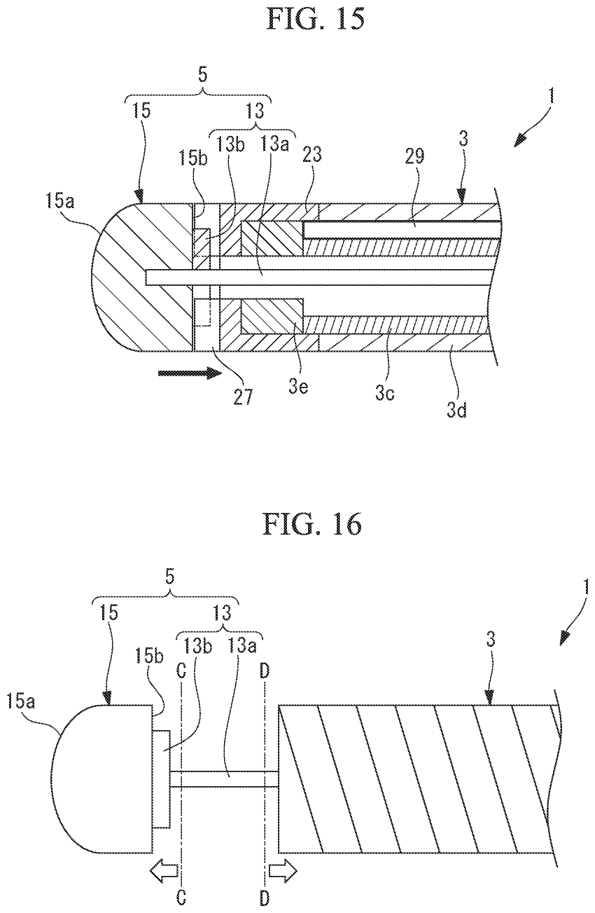

In this case, with this modification, the knife portion 5 is moved by means of the knife operating portion 6 in the direction in which the needle 13a is pulled into the sheath 3, as shown in FIG. 15. Accordingly, the burnt deposits of the biological tissue S remaining in a tubular shape around the needle 13a and the electrode 13b are pressed against the cutting edges 27a of the cutters 27 at the distal end of the sheath 3.

Then, as the needle 13a is pulled into the sheath 3, cuts are made in the burnt deposits of the biological tissue S in the longitudinal-axis direction of the needle 13a. Consequently, the needle 13a and the electrode 13b come off starting from the cuts in the burnt deposits of the biological tissue S and the burnt deposits of the biological tissue S peel off from the electrode portion 13.

Therefore, with the high-frequency treatment tool 1 according to this modification, it is possible to more efficiently remove the burnt deposits of the biological tissue S from the electrode portion 13.

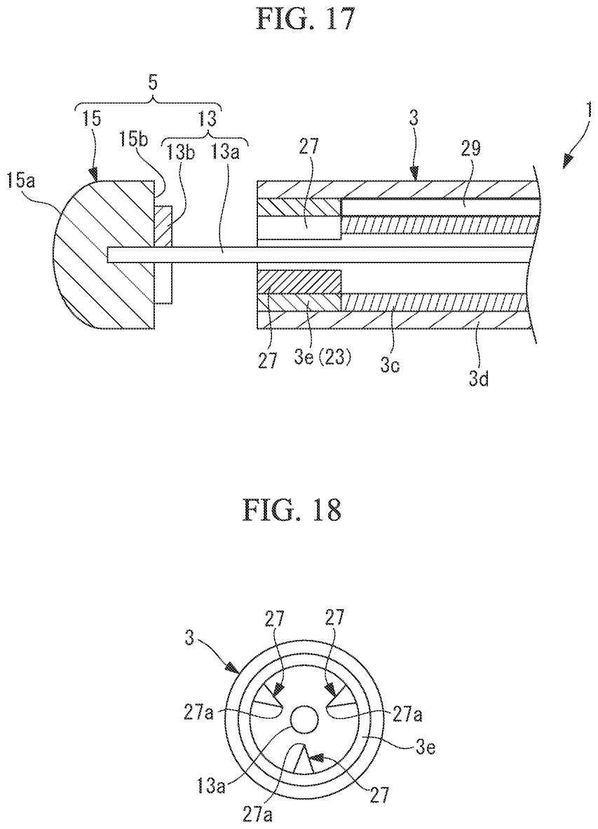

In this modification, the DC electrode 23 is disposed at the position where the DC electrode 23 covers the outer circumference of the distal-end portion of the sheath 3. Alternatively, for example, the DC electrode 23 may be accommodated in the distal-end portion of the sheath 3, as shown in FIGS. 16 and 17.

In the example shown in FIGS. 16 and 17, the tube 3d extends to the distal end of the sheath 3, and the cylindrical sheath distal-end member 3e disposed forward with respect to the coil 3c is covered with the tube 3d. In addition, the sheath distal-end member 3e is formed of a conductive material such as SUS and serves as the DC electrode 23.

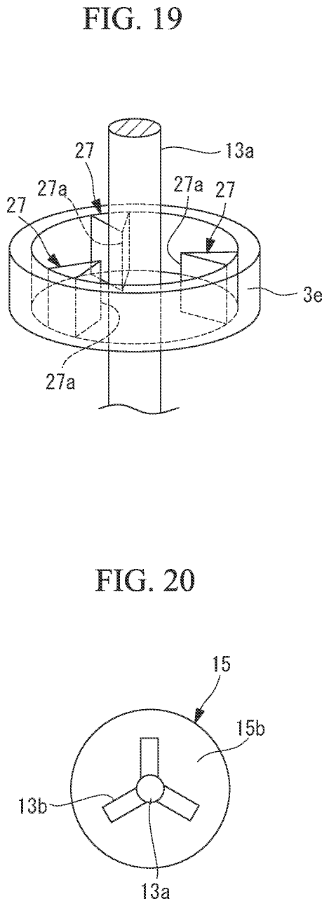

Each of the cutters 27 has, for example, the cutting edge 27a extending in the longitudinal direction of the sheath 3 and is secured to the inner surface of the sheath distal-end member 3e in an orientation in which the cutting edge 27a faces radially inward with respect to the sheath 3, as shown in FIGS. 18 and 19. In the example shown in FIGS. 18 and 19, three cutters 27 are disposed at positions shifted in the circumferential direction about the longitudinal axis of the needle 13a with respect to the electrode 13b extending in the Y-shape shown in FIG. 20.

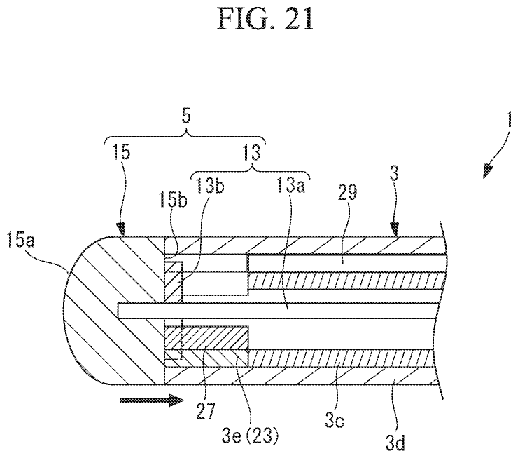

With this configuration also, as shown in FIG. 21, as a result of pulling the needle 13a into the sheath 3, burnt deposits of the biological tissue S remaining in a tubular shape around the needle 13a and the electrode 13b are pressed against the cutting edges 27a of the cutters 27 accommodated in the distal-end portion of the sheath 3, and thus, cuts are made in the burnt deposits of the biological tissue S. Therefore, it is possible to efficiently remove the burnt deposits of the biological tissue S from the electrode portion 13.

Although this modification has been described in terms of the three cutters 27 as an example, it suffices so long as cuts can be made in burnt deposits of the biological tissue S by means of the cutting edge 27a of the cutter 27, and the number of cutters 27 may be one, two, four, or more.

In this modification, the needle 13a is pulled into the sheath 3 after supplying direct currents between the needle 13a and the DC electrode 23 as well as between the electrode 13b and the DC electrode 23. Alternatively, direct currents may be supplied between the needle 13a and the DC electrode 23 as well as between the electrode 13b and the DC electrode 23 after making cuts in burnt deposits of the biological tissue S attached to the needle 13a and the electrode 13b by means of the cutting edges 27a of the cutters 27 by pulling the needle 13a into the sheath 3 first. In this case also, it is possible to efficiently remove the burnt deposits of the biological tissue S from the electrode portion 13.

As has been described above, although the embodiments of the present invention have been described in detail with reference to the drawings, specific configurations are not limited to said embodiments, and design alterations or the like within a range that does not depart from the scope of the present invention are also encompassed. For example, the present invention is not limited to application to the above-described respective embodiments and modifications, the present invention may be applied to embodiments in which said embodiments and modifications are combined, as appropriate, without particular limitation.

In addition, although the liquid has been described in terms of the physiological saline solution W as an example, any liquid may be employed so long as the liquid is an electrolyte liquid, and, for example, a liquid or the like present in biological tissue S may be utilized as the liquid. In addition, although the subject has been described in terms of a human as an example, the present invention may be applied to, for example, non-human animals. In addition, the attached matter has been described in terms of burnt deposits of biological tissue S as an example, it suffices so long as the attached matter can be peeled off from the electrode portion 13 by means of osmosis, and it is not limited to burnt deposits of biological tissue S.

The following aspects can be also derived from the embodiments.

A first aspect of the present invention is a high-frequency treatment tool including: a first electrode portion that is capable of applying a high-frequency current employed in high-frequency treatment; a second electrode portion that is disposed at a position at which the second electrode portion is electrically connected with the first electrode portion; and a power supply portion that uses the first electrode portion as a negative electrode, that uses the second electrode portion as a positive electrode, and that is capable of supplying a direct current between the first electrode portion and the second electrode portion.

With this aspect, it is possible to make a cautery incision in biological tissue by bringing the first electrode portion into contact with the biological tissue in a state in which the first electrode portion is energized with the high-frequency current.

In the case in which attached matter, such as a burnt deposit of the biological tissue (hereinafter, a burnt deposit of the biological tissue will be described as an example), becomes attached to the first electrode portion as a result of making a cautery incision in the biological tissue, the direct current is supplied between the first electrode portion and the second electrode portion by means of the power supply portion by using the first electrode portion as a negative electrode and by using the second electrode portion as a positive electrode. Consequently, a liquid moves due to osmosis, and, as a result of the liquid permeating the burnt deposit of the biological tissue and collecting in the periphery of the first electrode portion, it becomes easier for the burnt deposit of the biological tissue attached to the first electrode portion to peel off therefrom.

Therefore, in the case in which a burnt deposit of biological tissue becomes attached to the first electrode portion while treatment is being performed in a living body via an endoscope channel, it is possible to remove the burnt deposit of the biological tissue from the first electrode portion in a state in which the first electrode portion or the like remains inserted in the endoscope channel. Accordingly, even if a burnt deposit of biological tissue becomes attached to the electrode portion, it is possible to enhance the work efficiency by reducing the time and effort required to remove the high-frequency treatment tool from the endoscope channel.

In the above-described aspect, the high-frequency treatment tool may include a sheath having an inner hole that passes therethrough in a longitudinal direction, wherein the first electrode portion may be formed in a rod shape and may pass through the inner hole of the sheath to protrude from a distal end of the sheath.

In the above-described aspect, the second electrode portion may be an opposing electrode that is disposed outside a body of a subject and the high-frequency current may be supplied between the opposing electrode and the first electrode portion when an incision is made in biological tissue.

With this configuration, it is possible to share the second electrode portion between when making an incision in the biological tissue and when removing a burnt deposit of the biological tissue attached to the first electrode portion, and thus, it is possible to reduce the number of components.

In the above-described aspect, the second electrode portion may be a DC electrode that is disposed in a distal-end portion of the sheath and that is switched to an electrically non-contact state with respect to the first electrode when an incision is made in biological tissue.

In the case in which a burnt deposit of the biological tissue is removed with this configuration, because the direct current is concentrated in the periphery of the first electrode portion, it is possible to reduce the amount of current flowing inside the body.

In the above-described aspect, the high-frequency treatment tool may include a cutter that is disposed in a distal-end portion of the second electrode portion with a cutting edge thereof pointing toward the first electrode portion, wherein the first electrode portion may be provided so as to be relatively movable in the longitudinal direction in the inner hole of the sheath.

In the case in which a burnt deposit of the biological tissue attached to the first electrode portion is removed with this configuration, as a result of relatively moving the first electrode portion and the sheath in a direction in which the first electrode portion is pulled into the sheath after supplying the direct current between the first electrode portion and the second electrode portion by means of the power supply portion, the burnt deposit of the biological tissue attached to the first electrode portion is pressed against the cutting edge of a cutter disposed in the distal-end portion of the sheath. Accordingly, a cut is made in the burnt deposit of the biological tissue by means of the cutting edge of the cutter; therefore, it is possible to more efficiently remove the burnt deposit of the biological tissue from the first electrode portion.

In the above-described aspect, the sheath may include a coil that has the inner hole and that is formed of a tubular conductive material, a tube that covers an outer circumference of the coil and that is formed of an insulator, and a sheath distal-end member that is disposed forward with respect to the coil and the tube and that is formed of a tubular insulator; the second electrode portion may be formed in a tubular shape that covers a periphery of the sheath distal-end member; and the cutter may be disposed at a distal end of the second electrode portion.

In the above-described aspect, the sheath may include a coil that has the inner hole and that is formed of a tubular conductive material and a tube that covers an outer circumference of the coil and that is formed of an insulator; the second electrode portion may be formed in a tubular shape that is covered with the tube; and the cutter may be disposed on an inner surface of the second electrode portion.

In the above-described aspect, the power supply portion may supply, in a state in which an electrolyte liquid is interposed between the first electrode portion and the second electrode portion, the direct current between the first electrode portion and the second electrode portion via the liquid.

In the above-described aspect, the high-frequency treatment tool may include a liquid feeding means for supplying, as the liquid, a physiological saline solution between the first electrode portion and the second electrode portion.

With this configuration, as a result of facilitating the flow of the direct current between the first electrode portion and the second electrode portion via the physiological saline solution, it is possible to efficiently remove a burnt deposit of the biological tissue attached to the first electrode portion.

In the above-described aspect, the high-frequency treatment tool may include a switching mechanism that switches between energizing of the first electrode portion by means of the high-frequency current and energizing thereof by means of the direct current.

With this configuration, it is possible to switch, by means of the switching mechanism, the type of the current used to energize the first electrode portion between when making an incision in the biological tissue and when removing a burnt deposit of the biological tissue attached to the first electrode portion in a simple manner.

In the above-described aspect, the power supply portion may apply the high-frequency current and the direct current to the first electrode portion in an overlapping manner.

A second aspect of the present invention is a medical system including: any one of the high-frequency treatment tools described above; and an endoscope having a channel into which the high-frequency treatment tool can be inserted.

A third aspect of the present invention is a high-frequency treatment tool operating method in which: a first electrode portion is used as a negative electrode; a second electrode portion that is electrically connected with the first electrode portion is used as a positive electrode; and a direct current is supplied between the first electrode portion and the second electrode portion.

In the above-described aspect, after the direct current is supplied between the first electrode portion and the second electrode portion in a state in which the first electrode portion is disposed so as to protrude from a distal end of a sheath, the first electrode portion and the sheath may relatively be moved in a direction in which the first electrode portion is pulled into the sheath, and attached matter attached on the first electrode portion may be pressed against a cutting edge of a cutter disposed in a distal-end portion of the sheath.

In the above-described aspect, a physiological saline solution may be supplied between the first electrode portion and the second electrode portion.

### REFERENCE SIGNS LIST

1 high-frequency treatment tool
3 sheath
3a inner hole
3c coil
3d tube
3e sheath distal-end member
7 opposing electrode (second electrode portion)
11 liquid feeding means
13 electrode portion (first electrode portion)
19 constant-current DC power source (power supply portion)
21 switching mechanism
23 DC electrode (second electrode portion)
27 cutter
27a cutting edge
100 medical system
X patient (subject)
W physiological saline solution (liquid)

1. A high-frequency treatment tool comprising:
a sheath having an inner hole that passes therethrough in a longitudinal direction;
a first electrode portion that is formed in a rod shape, that passes through the inner hole of the sheath to protrude from a distal end of the sheath, and that is configured to apply a high-frequency current;
a second electrode portion that is disposed at a position at which the second electrode portion is electrically connected with the first electrode portion; and
a power source that uses the first electrode portion as a negative electrode, that uses the second electrode portion as a positive electrode, and that supply a current between the first electrode portion and the second electrode portion so that a state in which attached matter attached to the first electrode portion is lifted from the first electrode portion due to osmosis is created.

2. The high-frequency treatment tool according to claim 1, wherein the power source is configured to supply a direct current between the first electrode portion and the second electrode portion.

3. The high-frequency treatment tool according to claim 1, wherein the second electrode portion is an opposing electrode that is disposed outside a body of a subject and the high-frequency current is supplied between the opposing electrode and the first electrode portion when an incision is made in biological tissue.

4. The high-frequency treatment tool according to claim 2, wherein the second electrode portion is a DC electrode that is disposed in a distal-end portion of the sheath and that is switched to an electrically non-contact state with respect to the first electrode when an incision is made in biological tissue.

5. The high-frequency treatment tool according to claim 4, further comprising a cutter that is disposed in a distal-end portion of the second electrode portion with a cutting edge thereof pointing toward the first electrode portion, wherein
the first electrode portion is provided so as to be relatively movable in the longitudinal direction in the inner hole of the sheath.

6. The high-frequency treatment tool according to claim 5, wherein:
the sheath includes a coil that has the inner hole and that is formed of a tubular conductive material, a tube that covers an outer circumference of the coil and that is formed of an insulator, and a sheath distal-end member that is disposed forward with respect to the coil and the tube and that is formed of a tubular insulator;
the second electrode portion is formed in a tubular shape that covers a periphery of the sheath distal-end member; and
the cutter is disposed at a distal end of the second electrode portion.

7. The high-frequency treatment tool according to claim 5, wherein:
the sheath includes a coil that has the inner hole and that is formed of a tubular conductive material and a tube that covers an outer circumference of the coil and that is formed of an insulator;
the second electrode portion is formed in a tubular shape that is covered with the tube; and
the cutter is disposed on an inner surface of the second electrode portion.

8. The high-frequency treatment tool according to claim 1, wherein the power source supplies, in a state in which an electrolyte liquid is interposed between the first electrode portion and the second electrode portion, the current between the first electrode portion and the second electrode portion via the liquid.

9. The high-frequency treatment tool according to claim 8, further comprising a feeder that supply, as the liquid, a physiological saline solution between the first electrode portion and the second electrode portion.

10. The high-frequency treatment tool according to claim 2, further comprising a switch that switches between energizing of the first electrode portion by means of the high-frequency current and energizing thereof by means of the direct current.

11. The high-frequency treatment tool according to claim 2, wherein the power source applies the high-frequency current and the direct current to the first electrode portion in an overlapping manner.

12. A medical system comprising:
A high-frequency treatment tool according to claim 1; and
an endoscope having a channel into which the high-frequency treatment tool can be inserted.

13. A method for removing attached matter on a high-frequency treatment tool, the method comprising:
making a first electrode portion disposed in a sheath protrude from a distal end of the sheath toward a distal end, the first electrode portion being formed in a rod shape;
releasing an electrolyte liquid from the distal end of the sheath toward the first electrode portion;
supplying a current between the first electrode portion and a second electrode portion so that a state in which attached matter attached to the first electrode portion is lifted from the first electrode portion due to osmosis is created, the second electrode portion being disposed at a position at which the second electrode portion is electrically connected with the first electrode portion; and
pulling the first electrode portion into the sheath.

14. The method according to claim 13, wherein in the supplying, a direct current is supplied between the first electrode portion and the second electrode portion.

15. The method according to claim 13, wherein:
the supplying is performed after the making and the releasing; and
in the supplying, in a state in which the liquid is interposed between the first electrode portion and the second electrode portion, supplying a direct current between the first electrode portion and the second electrode portion via the liquid.

16. The method according to claim 13, wherein in the pulling, the attached matter attached to the first electrode portion is pressed against a cutter provide at the distal end of the sheath.

17. The method according to claim 13, further comprising supplying a high-frequency current between the first electrode portion and the second electrode portion to make an incision in biological tissue by the first electrode portion.
